(19) United States
(12) Patent Application Publication
(10) Pub. No.: US 2021/0299167 A1
(43) Pub. Date: Sep. 30, 2021

(54) STABLE IODINE-CONTAINING ANTIMICROBIAL TEAT DIP COMPOSITIONS

(71) Applicant: ECOLAB USA INC., St. Paul, MN (US)

(72) Inventors: Jinsen GAO, Shanghai (CN); Yan ZHENG, Shanghai (CN); Colin COURT, Castle Hill (AU); Shahar RONEN, East Ryde (AU)

(21) Appl. No.: 17/346,615

(22) Filed: Jun. 14, 2021

Related U.S. Application Data
(63) Continuation-in-part of application No. PCT/CN2018/121124, filed on Dec. 14, 2018.

(51) Int. Cl. A61K 33/18; A61K 47/34; A61K 47/20; A61K 9/00

(52) U.S. Cl. CPC A61K 33/18; A61K 9/0014; A61K 47/20; A61K 47/34

(57) ABSTRACT

The germicidal aqueous teat dip compositions contain a stabilizing amount of a EO-PO copolymer composition and an antimicrobial effective amount of iodine. The iodine-based teat dip compositions can be used to reduce the incidence of contagious and environmental mastitis in dairy heads. The compositions can be applied to udder and teats of dairy herds to form a protective coating that can prevent infection from Staphylococcus, Streptococcus, Klebsiella, and other pathogens.

This application is a Continuation-in-Part of PCT/CN2018/121124, filed on Dec. 14, 2018, the disclosure of which is incorporated herein by reference in its entirety. To the extent appropriate, a claim of priority is made to the above disclosed application.

### TECHNICAL FIELD

The present disclosure relates to compositions used in the treatment or prevention of environmental and contagious mastitis in mammals, such as cows, undergoing common milking operations. Specifically, embodiments of the present disclosure relate to aqueous iodine-based teat dips which include a stabilizing copolymer composition containing a mixture of poly(ethylene oxide)-poly(propylene oxide)-poly(ethylene oxide) block copolymers.

### BACKGROUND TECHNOLOGY

The effective management and maintenance of large dairy herds and the production of dairy products has been a major agricultural accomplishment. One of the problems in maintaining large herds is the health of the individual animals. One health problem in individual animals of dairy herds that causes significant economic problems relates to mastitis. Often during milking, the animals skin is irritated by automated milking machines. A typical milking machine mechanically simulates the hand milking of a cow through the use of a pulsating vacuum. For example, a vacuum pump is attached to the teat, and the vacuum is then pulsated to alternately allow the milk to fill and drain from the area of the udder and teat. Milking times may be reduced by increasing the vacuum strength, however, this practice causes irritation or damage to the teat and udder. Exposure of damaged tissue to certain microorganisms can result in an infection known as mastitis. Animals that contract mastitis must be removed from service resulting in the loss of dairy output. As a result, a significant amount of attention has been focused on preventing the development of mastitis or treating mastitis in dairy herds.

The dairy farmer is generally faced with two different types of mastitis infections. Contagious mastitis is spread during the milking process through contact between the animal and dairy equipment that may carry a mastitis pathogen. Contagious mastitis is most easily controlled by using antimicrobial post milking teat dips. Such antimicrobial teat dips kill the bacteria that are introduced onto the surface of the animal from the milking machines. The second type of mastitis, environmental mastitis, is caused by contamination of the animal surface by materials from the barn yard environment, fields, barn interior, etc. Such pathogens include E. coli, Streptococcus uberis, Klebsiella and others. Such contamination occurs as the animal moves through its environment. Environmental mastitis is best treated with a barrier film that protects sensitive tissues from contamination.

Many teat dip products consist of an effective antimicrobial amount of iodine. Elemental iodine is considered a broad-spectrum germicide that is capable of rapid and complete killing of bacteria on contact. Iodine solubility in water, however, is generally low. Therefore, aqueous iodine formulations require the presence of carrier molecules such as surfactants, glycols, or polyvinyl pyrrolidone (PVP), which solubilize the iodine through complexation. Generally referred to as iodophors, these iodine-carrier molecule complexes can consist of up to 20% iodine by weight.

Previous examples of iodine-based teat dips have utilized a variety of surfactants as carrier molecules. Among the most favored complexing agents described are nonylphenol ethoxylate (“NPE”) or its derivatives and linear alcohol ethoxylates (“LAE”) and their derivatives. In recent years, however, NPE has been shown to be toxic to aquatic life and also identified as endocrine disrupters. These findings have led to concerns over NPE contamination in milk consumed by humans. In fact, the European Union has placed restrictions on the presence of NPE in various compositions including teat dips. Under these regulations the concentration of NPE in the composition must be less than 0.1 wt. %. Like NPE, linear alcohol ethoxylates (“LAE”) derivatives have fallen under scrutiny for their potential toxicity. Several countries have banned its use in agricultural products over these concerns.

In light of the recent revelations regarding the toxicity of NPEs and the potential health risks associated with exposure to LAEs, there remains a need for the development of iodine-based teat dip compositions containing a stabilized iodine composition in the absence of NPE, NPE derivatives, LAE, and LAE derivatives.

### SUMMARY OF THE INVENTION

The present disclosure generally relates to iodine-based antimicrobial compositions that exhibit time and temperature stability in the absence of NPE and LAE. The antimicrobial compositions of the present disclosure are suitable for a broad range of applications, but have been found to be particularly useful for application to the teats and udders of dairy animals as udder and teat washes, and as pre-milking and post-milking sanitizing solutions.

The stable iodine-based teat dip compositions of the present disclosure comprise of a poly(ethylene oxide)-poly(propylene oxide)-poly(ethylene oxide) block (“EO-PO”) copolymer composition comprising of a first EO-PO copolymer component and a second EO-PO copolymer component, in which the iodine-based teat dip composition is substantially free of NPE and LAE.

Preferred embodiments include stable iodine-based teat dip compositions comprising of a first EO-PO copolymer component and a second EO-PO copolymer component such that a first EO-PO copolymer component has a higher number average molecular weight than a second EO-PO copolymer component. In other embodiments the number average molecular weight of the first EO-PO copolymer component is at least 300 higher than the second EO-PO copolymer component. In other embodiments the number average molecular weight of the first EO-PO copolymer component is at least 1000 higher than the second EO-PO copolymer component. In still yet other embodiments the number average molecular weight of the first EO-PO copolymer component is at least 2500 higher than the second EO-PO copolymer component.

In some embodiments of the present disclosure the first EO-PO copolymer component has a number average molecular weight of 3600 or above and the second EO-PO copolymer component has a number average molecular weight below 3600. In other embodiments of the present disclosure, the first EO-PO copolymer component has a number average molecular weight in the range from about 3600 to about 12,600; and a stabilizing amount of a second EO-PO copolymer component having a number average molecular weight in the range from about 1000 up to about 3600.

In some embodiments of the present disclosure the stable iodine-based teat dip compositions comprise of a EO-PO copolymer composition comprising of a first EO-PO copolymer component and a second EO-PO copolymer component; a hydrotrope, and an antimicrobial effective amount of iodine, wherein the stable iodine composition is completely free of NPE and LAE.

In some embodiments of the present disclosure the stable iodine compositions comprise of a EO-PO copolymer composition comprising of a first EO-PO copolymer component and a second EO-PO copolymer component wherein the first EO-PO copolymer component has a number average molecular weight at least 1000 higher than the second EO-PO copolymer component; a hydrotrope, and an antimicrobial effective amount of iodine, wherein the stable iodine composition is completely free of NPE and LAE.

In some embodiments of the present disclosure the stable iodine compositions comprise of a EO-PO copolymer composition comprising of a first EO-PO copolymer component having a number average molecular weight of 3600 to 12,000, and a second EO-PO copolymer component having a number average molecular weight of less than 3600; sodium xylene sulfonate, and an antimicrobial effective amount of iodine, wherein the stable iodine composition is essentially completely free of NPE and LAE.

In some embodiments of the present disclosure the stable iodine compositions comprise of a EO-PO copolymer composition comprising of a first EO-PO copolymer component having a number average molecular weight of 5600 and a second EO-PO copolymer component having a number average molecular weight of 2900, sodium xylene sulfonate, and an antimicrobial effective amount of iodine, wherein the stable iodine composition is substantially free of NPE and LAE.

### BRIEF DESCRIPTION OF THE FIGURES

FIG. 1 shows a contour plot of the effect of a stabilizing concentration of the EO-PO copolymer composition on iodine availability in iodine-based teat dip compositions after storage for 3 weeks at 50° C.

FIG. 2 shows the effects of sodium xylene sulfonate on available iodine after prolonged storage at 50° C. in concentrated iodine-based teat dip compositions containing 2 wt. % iodine and an EO-PO copolymer composition comprising of 4.0 wt. % P104 and 4.0 wt. % PE6400.

FIG. 3 shows the impact of pH on available iodine after prolonged storage at 50° C. in ready-to-use iodine-based teat dip compositions containing 0.5 wt. % iodine and a EO-PO copolymer composition comprising of 1.0 wt. % P104 and 1.0 wt. % PE6400.

FIG. 4 shows the availability of iodine after prolonged storage at both 40° C. and 50° C. for concentrated iodine-based teat dip compositions containing 2 wt. % iodine and NPE.

FIG. 5 shows the availability of iodine after prolonged storage at both 40° C. and 50° C. for ready-to-use iodine-based teat dip compositions containing 0.5 wt. % iodine and NPE.

### DETAILED DESCRIPTION OF THE INVENTION

Unless otherwise specified, the following terms as used herein have the meanings provided below.

“Killing” as the term is used herein includes actual killing as well as inhibition or abatement of microorganism growth.

The term “substantially free” of a particular substance means that the compositions of the instant specification contain less than 0.5 wt. % of the recited substance. When referring to “substantially free” it is intended that the substance is not intentionally added to the compositions. The term “essentially free” of a particular substance means that the compositions of the instant specification contain less than 0.1 wt. % of the recited substance. When referring to “essentially free” it is intended that the substance is not intentionally added to the compositions. The term “essentially completely free” of a particular substance means that the compositions of the instant specification contain less than 0.01 wt. % of the recited substance. When referring to “essentially completely free” it is intended that the substance is not intentionally added to the compositions. The term “completely free” of a particular substance means that the compositions of the instant specification contain less than 0.001 wt. % of the recited substance. When referring to “completely free” it is intended that the substance is not intentionally added to the compositions. Use of the term “completely free” allows for trace amounts of that substance to be included in compositions because they are present in another substance in the composition. However, it is recognized that only trace or de minimus amounts of a substance will be allowed when the composition is said to be “completely free” of that substance.

The term “about” generally refers to a range of numbers that one of skill in the art would consider equivalent to the recited value (i.e., having the same function or result). In many instances, the terms “about” may include numbers that are rounded to the nearest significant figure.

As used herein, the singular forms “a”, “an”, and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly dictates otherwise.

Weight percent, percent by weight, wt. %, and the like are synonyms that refer to the concentration of a substance as the weight of that substance divided by the weight of the composition and multiplied by 100.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein the term “comprising” and variations thereof do not have a limiting meaning where these terms appear in the specification and claims.

As used herein the term, “consisting essentially of” in reference to a composition refers to the listed ingredients and does not include additional ingredients that, if present, would affect the composition. The term “consisting essentially of” may also refer to a component of the composition.

As used herein the term “NPE” refers to nonylphenol ethoxylate and its derivatives. NPE is a class of nonionic surfactants including the condensation products of one mole of alkyl phenol wherein the alkyl constituent contains from about 8 to about 18 carbon atoms with from about 3 to about 50 moles of ethylene oxide. The alkyl group can, for example, be represented by diisobutylene, di-amyl, polymerized propylene, isoctyl, nonyl, and di-nonyl. Examples of commercial compounds of this chemistry are available on the market under the trade name IGEPAL® manufactured by Rhone-Poulenc.

As used herein the term “LAE” refers to linear alcohol ethoxylates. LAE are a class of nonionic surfactants that contain a hydrophobic alkyl chain attached via an ether linkage to a hydrophilic ethylene oxide (EO) chain and have the general structure R(OCH₂CH₂)ₙOH. The alkyl chain, R, can vary in length and in the degree of linearity, but is typically between 8 and 18 carbons long. The EO chain can also vary in length from 1 to 40 EO units. A LAE with the structure C₉₋₁₁ EO 6.5, for example, contains a range of alkyl chain lengths of 9-11 and averages 6.5 EO units per alkyl chain.

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 supports claims to any of the following ranges: 1-5; 1-4; 1-3; 1-2; 2-5; 2-4; 2-3; 3-5; 3-4; and 4-5.

In order to be dependable and useful to an end user, iodine-based teat dip compositions must be stable (i.e., remain homogeneous) over a wide range of temperatures. If stability is lost, and the composition separates, the utility of the composition is significantly degraded and can present a potential hazard to the user. Generally speaking, stability in this context means that a given composition must remain homogeneous after extended storage (e.g., 1 week or more) at temperatures as low as 4° C. (which may be experienced in cold warehouse storage areas) or as high as 50° C. (which can occur during transport in closed vehicles). Another desirable functional characteristic for iodine-based teat dip compositions designed for topical application, is the ability to spread evenly on the skin and not drain off so rapidly as to prevent insufficient germicidal contact time. Elemental iodine is highly insoluble in aqueous solutions. Therefore, in order to use iodine in aqueous formulations elemental iodine is usually complexed with certain carrier molecules such as surfactants, glycols, or polyvinyl pyrrolidone (PVP). Surprisingly, it has been shown that stable iodine-based teat dip compositions are provided by a copolymer composition containing a mixture of poly(ethylene oxide)-poly(propylene oxide)-poly(ethylene oxide) block copolymers (“EO-PO copolymers”). The compositions of the present disclosure have unique properties which make them superior to other iodine-based teat dips. These include shelf stability and iodine stability over a large range of temperatures.

These EO-PO copolymers are generically referred to as poloxamers. Poloxamers are tri-block copolymers, of the formula A-B-A or B-A-B, where A is poly(ethylene oxide) and B is poly(propylene oxide), as scene in Scheme 1.

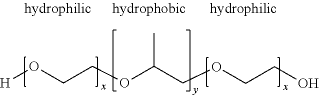

EO-PO copolymers are formed by condensing ethylene oxide with a hydrophobic base formed by the addition of propylene oxide to two hydroxyl groups of propylene glycol. Ethylene oxide is then added to sandwich this hydrophobe between hydrophilic groups. The monomers comprising the copolymer blocks of these EO-PO copolymers are chemically dissimilar (e.g. polar and nonpolar), causing them to have interesting surface activity features. For instance, EO-PO copolymers exhibit an amphiphilic character in aqueous solution on the basis of ethylene oxide solubility in water and polypropylene oxide insolubility in water. Stated another way, the EO blocks are hydrophilic, while the PO block is hydrophobic. This block segregation gives rise to useful nanostructures such as micelle formation. The size and structure of copolymer assemblies, and their adsorption properties have made them useful in many applications including, drug delivery, nanoparticle synthesis, and cosmetics.

Examples of commercially available EO-PO copolymers are PLURONIC®, PLURONIC® PE, and PLURACARE® copolymers manufactured by BASF Corp and described generally in U.S. Pat. No. 3,740,421 issued to Schmolka et al. EO-PO copolymer nomenclature varies depending on the commercial source. The naming convention for generic EO-PO copolymers includes a letter “P” with a two or three digit number, wherein the first two digits multiplied by 100 indicate the approximate molecular mass of the polypropylene oxide block, and the last digit multiplied by 10 gives the approximate percentage of polyethylene oxide block (e.g. P 407 is a EO-PO copolymer with a polypropylene oxide molecular mass of approximately 4000 g/mol and a 70% polyethylene oxide content). PLURONIC® EO-PO copolymers use a different naming convention from the generic convention with an alphabetical letter indicating the physical state (P: Paste, F: Flake, L: Liquid) of the copolymer and a two or three digit number; the first digit—or first two digits in a three-digit number-multiplied by 300 indicates the approximate molecular weight of the polypropylene oxide block, while the last digit multiplied by 10 provides approximate percentage of polyethylene oxide block (e.g. L61 indicates a EO-PO copolymer that is liquid with an approximate molecular mass of polypropylene oxide block of 1800 g/mol and approximately 10% polyethylene oxide content). PLURONIC® PE copolymers are designated by a letter and a four or five-digit number; the first digit is guide to the approximate molar mass of the polypropylene oxide block on a scale of 1-10. The second, or third, digit divided by 10 provides the percentage of polyethylene oxide. For instance, PLURONIC® PE3100 has a polypropylene oxide block with an approximate molar mass of 850 g/mol and contains approximately 10% polyethylene oxide, whereas PLURONIC® PE10100 has a polypropylene oxide block with an approximate molar mass of 3250 g/mol and contains approximately 40% polyethylene oxide. PLURACARE® copolymers are designated by a letter, indicating the physical state of the copolymer and a three digit number. For example, PLURACARE® F127 is a flake copolymer where the first two digits multiplied by 300 indicates the approximate molar mass of the polypropylene oxide block and the last digit, when multiplied by 10 indicates the approximate polyethylene oxide content of the copolymer.

Most commercial sources of EO-PO copolymers provide information regarding the number average molecular weight of the entire copolymer. The number average molecular weight can be determined by various methodologies such as fractionation, ultracentrifugation analysis, as well as, the mathematical analysis as described in FLORY, P. J. J. Am. Chem. Soc. 62, pg. 1501-04. In other instances, only the molar mass of the hydrophobe or central polypropylene oxide block is provided. For simplification the molecular weights of the EO-PO copolymers described in the present disclosure are their number average molecular mass.

Examples of preferred EO-PO copolymers for the embodiments of the present disclosure include but are not limited to the following EO-PO copolymers available from BASF: PE3100, PE6200, PE6400, PE9400, PE10100, P104, P105, F127, F108. These EO-PO copolymers are of the A-B-A type shown in scheme 1 and are available in a broad range of number average molecular weights and EO/PO ratios.

Not wishing to be bound by theory it is believe that the EO-PO copolymer composition forms a stable iodine-based teat dip composition by creating a physical barrier or micelle which separates the iodine particles allowing them to stay in solution. The hydrophobic polypropylene oxide block adsorbs on the iodine particles while the long hydrophilic polyethylene oxide block tail orients towards the water phase. Lengthening the hydrophilic tail causes greater extension of the shear plane and results in improved particle protection. Further, a long propropylene oxide chain can provide multiple points of attachment to the particle, making desorption less likely than for conventional nonionic surfactant (which have only a single hydrophobe). The twin polyethylene oxide hydrophiles offer extra stabilization for a lower surfactant concentration and longer polyethylene oxide chains extend the shear plane further than other nonionic surfactants thus improving dispersion stability.

Preferred EO-PO copolymers for the stable iodine-based teat dip compositions of the present disclosure are chosen such that the polyethylene oxide block constituent comprises from about 10 wt. % to about 90 wt. %, from about 20 wt. % to about 70 wt. %, from about 30 wt. % to about 50 wt. %, from about 40 wt. % to about 60 wt. %, or from about 50 wt. % to about 80 wt. % of the total copolymer. The preferred EO-PO copolymers have a number average molecular weight of from about 1,000 to about 15,000, from about 2,000 to about 10,000, from about 4,000 to about 8,000, from about 5,000 to about 7,000, from about 2900 to about 5,900, and from about 1,000 to about 3,600.

Some embodiments include a stable iodine-based teat dip compositions comprising of an EO-PO copolymer composition comprising of a first EO-PO copolymer component and a second EO-PO copolymer component such that the first EO-PO copolymer component has a number average molecular weight that is higher than the second component. In some embodiments the number average molecular weight of the first EO-PO copolymer component is at least 300, at least 500, at least 700, at least 1000, at least 1500, at least 2,000 or at least 5000 higher than the number average molecular weight of the second EO-PO copolymer component.

In some embodiments the first EO-PO copolymer component has a number average molecular weight from about 3,000 to about 15,000, from about 3,600 to about 12,600, from about 4,500 to about 10,000, from about 5,000 to 7,000, or from about 5,900 to about 6500.

In some embodiments the second EO-PO copolymer component has a number average molecular weight from about 1,000 to about 4,000, from about 1,000 to about 3,600, from about 1,000 to about 2,900, or from about 500 to about 2,000.

In some embodiments, the stable iodine composition has a weight ratio between the first EO-PO copolymer component and the second EO-PO copolymer component range from about 1:10 to about 10:1, from about 2:8 to about 8:2, from about 3:2, about 2:3, from about 0.5:1 to about 1:0.5, or from about 0.01:1 to about 1:0.01. In some embodiments the weight ratio between the first EO-PO copolymer component and the second EO-PO copolymer is about 1:1.

In some embodiments the first EO-PO copolymer component is present in the range of about 0.1 wt. % to about 10 wt. %, about 1.0 wt. % to about 8 wt. %, about 2 wt. % to about 6 wt. %, about 3 wt. % to about 5 wt. %, or about 4 wt. % of the total composition. In some embodiments the second EO-PO copolymer component is present in the range of about 0.1 wt. % to about 10 wt. %, about 1.0 wt. % to about 8 wt. %, about 2 wt. % to about 6 wt. %, about 3 wt. % to about 5 wt. % of the total composition, or about 4 wt. % of the total composition.

In some embodiments the total amount of EO-PO copolymer present in the iodine-based teat dip composition is in the range from about 1.0 wt. % to about 20 wt. %. In other embodiments the total EO-PO copolymer present in the composition is in the range from about 1.0 wt. % to about 9.0 wt. %, from about 1.0 wt. % to about 4.0 wt. %, from about 8.0 wt. % to about 20 wt. %, from about 4.0 wt. % to about 14.0 wt. %, from about 5.0 wt. % to about 8.0 wt. %, or from about 0.5 wt. % to about 2.0 wt. %. These ranges can refer to a ready-to-use composition or a concentrate depending on the desired properties of the final teat dip composition.

In addition to the EO-PO copolymer components, the iodine-based teat dip compositions are completely free, essentially completely free, or substantially free from NPE or nonionic surfactants that include the condensation products of one mole of alkyl phenol wherein the alkyl constituent contains from about 8 to about 18 carbon atoms with from about 3 to about 50 moles of ethylene oxide. Examples of commercial compounds of this chemistry are available on the market under the trade name IGEPAL® manufactured by Rhone-Poulenc and TRITON® manufactured by Dow. Particularly, the compositions of the present disclosure are substantially free of nonyl phenol ethoxylate 12 mole (NPE 12), or IGEPAL® CO-720 available from Rhone-Poulenc. The compositions contain no more than 0.1 wt. % of NPE, no more than 0.01 wt. % of NPE, or no more than 0.001 wt. % NPE of the total ready to use composition.

In addition to the EO-PO copolymer components, the iodine-based teat dip compositions are completely free, essentially completely free, or substantially free from LAE, where LAE is a class of nonionic surfactants that contain a hydrophobic alkyl chain attached via an ether linkage to a hydrophilic ethylene oxide (EO) chain and have the general structure R(OCH₂CH₂)ₙOH. The compositions contain no more than 0.1 wt. % of LAE, no more than 0.01 wt. % of LAE, or no more than 0.001 wt. % LAE of the total ready to use composition.

### Iodine Compounds

In some embodiments of the present disclosure, the iodine can be provided as an activated iodine premix, for example, NaOH/I₂ or NaI/I₂. The iodine premix can be formulated by adding deionized water, iodine and an iodide constituent to a reactor with adequate mixing. Generally, the iodide constituent can be any alkaline earth metal-iodine salt such as sodium iodide or potassium iodide. An NaI/I₂ premix at a concentration of about 1.8% of the total weight of the use solution provides about 1% titratable I₂ in the use solution. In other embodiments the iodine is provided by adding deionized water, iodine and sodium hydroxide to a reactor with adequate mixing. An NaOH/I₂ premix at a concentration of about 1.8% of the total weight of the use solution provides about 1% titratable 12 in the use solution. The activated iodine premix is added to the EO-PO copolymer composition. Preferred embodiments provide a titratable iodine concentration from about 0.05% to about 5% wt. %, from about 1.0 to about 3.0 wt. %, from about 1.5 to about 2.5 wt. %, from about 2.0 wt. % to about 2.5 wt. %, or from about 0.3 wt. % to about 1.0 wt. %. These ranges can refer to a ready-to-use composition or a concentrate depending on the desired properties of the final teat dip composition.

### Hydrotropes

In certain embodiments it has been found that the addition of solubilizing agents called hydrotropes or couplers are preferred. Hydrotropes may be used to maintain physical single phase integrity and storage stability. To this end, any number of ingredients known to those skilled in the art may be employed, such as monofunctional and polyfunctional alcohols. These preferably contain from about 1 to about 6 carbon atoms and from 1 to about 6 hydroxy groups. Examples include ethanol, isopropanol, n-propanol, 1,2-propanediol, 1,2-butanediol, 2-methyl-2,4-pentanediol, mannitol and glucose. Also useful are the higher glycols, polyglycols, polyoxides, glycol ethers and propylene glycol ethers. Additional useful hydrotropes include the free acids and alkali metal salts of sulfonated alkylaryls such as toluene, xylene, cumene and phenol or phenol ether or diphenyl ether sulfonates; alkyl and dialkyl naphthalene sulfonates and alkoxylated derivatives. Examples of preferred hydrotropes are xylene sulfonate, 1-octane sulfonate and 1,2-octane disulfonate.

Preferred hydrotropes for the iodine-based teat dip compositions include sodium cumene sulphate or sodium xylene sulfonate (“SXS”). SXS is found in personal care products because of its ability to serve as a claritant or wetting agent that helps a formula spread more easily. In some embodiments, the hydrotrope provides high temperature stability and cloud point control. Generally, the hydrotrope component may be present from about 0.01 wt. % to about 10 wt. %, from about 0.1 wt. % to about 10 wt. %, from about 1.0 wt. % to about 10 wt. %, from about 4.0 wt. % to about 10 wt. %, from about 2% to about 8%, or from about 1.5 wt. % to about 4.0 wt. % of the composition. These ranges can refer to a ready-to-use composition or a concentrate composition depending on the desired properties of the final teat dip composition.

### Surfactants/Wetting Agents

Although EO-PO copolymers exhibit nonionic surfactant properties the presence of additional wetting agents or surfactants have been found to be beneficial for the iodine teat dip compositions of the present disclosure. Wetting agents such as surfactants may be included to formulate the disclosed compositions for an intended environment of use. Typical wetting agents are used to wet the surface of application, reduce surface tension of the surface of application so that the product can penetrate easily on the surface and remove unwanted soil. The wetting agents or surfactants of the formulation increase overall detergency of the formula, solubilize or emulsify some of the organic ingredients that otherwise would not dissolve or emulsify, and facilitate penetration of active ingredients deep onto the intended surface of application, such as animal skin.

Suitable surfactants may include but are not limited to anionic, cationic, nonionic, zwitterionic and amphoteric surfactants. Suitable anionic surfactants can be chosen from alkyl sulfonic acid, alkyl sulfonate salt, alkyl sulfate salt, linear alkylbenzene sulfonic acid, a linear alkylbenzene sulfonate salt, alkyl α-sulfomethyl ester, alkyl α-olefin sulfonate salt, alcohol ether sulfate salt, alkyl sulfate salt, alkylsulfo-succinate salt, a dialkylsulfosuccinate salt, and their alkali metal, alkaline earth metal, amine and ammonium salts thereof. Specific examples are linear C₁₀-C₁₆ alkylbenzene sulfonic acid, linear C₁₀-C₁₆ alkylbenzene sulfonate or alkali metal, alkaline earth metal, amine, alkanol amine and ammonium salts thereof, e.g., sodium xylene sulfonate, sodium dodecylbenzene sulfonate, sodium octane sulfonate, sodium lauryl sulfate, sodium C₁₄-C₁₆ α-olefin sulfonate, C₁₂-C₁₈ sodium methyl α-sulfomethyl ester and C₁₂-C₁₈ disodium methyl α-sulfo fatty acid salt. Suitable nonionic surfactants can be chosen from alkyl polyglucoside, alkyl ethoxylated alcohol, alkyl propoxylated alcohol, ethoxylated propoxylated alcohol, sorbitan, sorbitan ester and alkanol amide. Specific examples include C₈-C₁₆ alkyl polyglucoside with a degree of polymerization ranging from 1 to 3, e.g., C₈-C₁₀ alkyl polyglucoside with a degree of polymerization of 1.5 (Glucopon® 200), C₈-C₁₆ alkyl polyglucoside with a degree of polymerization of 1.45 (Glucopon® 425), C₁₂-C₁₆ alkyl polyglucoside with a degree of polymerization of 1.6 (Glucopon® 625). Other useful nonionic surfactants include condensation products of one mole of saturated or unsaturated, straight or branched chain carboxylic acid having from about 8 to about 18 carbon atoms with from about 6 to about 50 moles of ethylene oxide. The acid moiety can consist of mixtures of acids in the above delineated carbon atom range or it can consist of an acid having a specific number of carbon atoms within the range. Examples of commercial compounds of this chemistry are available on the market under the trade name NOPALCOL® manufactured by Henkel Corporation and LIPOPEG® manufactured by Lipo Chemicals, Inc. In addition to ethoxylated carboxylic acids, commonly called polyethylene glycol esters, other alkanoic acid esters formed by reaction with glycerides, glycerin, and polyhydric (saccharide or sorbitan/sorbitol) alcohols can be used in the composition. All of these ester moieties have one or more reactive hydrogen sites on their molecule which can undergo further acylation or ethylene oxide (alkoxide) addition to control the hydrophilicity of these substances.

Amphoteric surfactants can be chosen from alkyl betaines, alkylamido betaines, alkylamidoalkyl betaines and alkyl amphoacetates. Suitable betaines include cocoamidopropyl betaine, and suitable amphoacetates include sodium cocoamphoacetate, sodium lauroamphoacetate and sodium cocoamphodiacetate.

### Thickening Agents

A composition of the present disclosure may optionally contain one or more rheology modifiers, to enhance viscosity, or thicken the composition to facilitate adherence of a dip to the teat. Adherence enables the composition to remain in contact with transient and resident pathogenic bacteria for longer periods of time, promoting microbiological efficacy and resisting waste because of excessive dripping. The rheology modifier may be a film former or act cooperatively with a film-forming agent to form a barrier that provides additional protection. An important aspect of this thickening is that the iodine teat dip composition is fluid enough for pouring or dipping but still has sufficient thixotropy or viscosity to resist rapid draining or running off from the teat or udder.

A variety of organic and inorganic agents can increase the viscosity, apparent viscosity, or shear-dependent viscosity (thixotropy) of water. Preferred aqueous thickening agents which are more useful in the compositions of the present disclosure are those which are non-Newtonian (psuedoplastic) that tend not to develop rigid intermolecular interactions. Inorganic thickeners are generally compounds such as colloidal magnesium aluminum silicate (VEEGUM®), colloidal clays (Bentonites), or silicas (CAB-O-SILS®) which have been fumed or precipitated to create particles with large surface to size ratios. Suitable natural hydrogel thickeners are primarily vegetable derived exudates, e.g., tragacanth, karaya, and acacia gums; extractives, e.g., caragheenan, locust bean gum, guar gum and pectin; or pure culture fermentation products such as xanthan gum. Chemically, all of these materials are salts of complex anionic polysaccharides. Synthetic natural-based thickeners having application are cellulosic derivatives wherein the free hydroxyl groups on the linear anhydro-glucose polymers have been etherified or esterified to give a family of substances which dissolve in water and give viscous solutions. This group of materials includes the alkyl and hydroxylalkycelluloses, specifically methylcellulose, hydroxyethylmethylcellulose, hydroxypropylmethylcellulose, hydroxybutylmethylcellulose, hydroxyethylcellulose, ethylhydroxyethylcellulose, hydroxypropylcellulose, and carboxymethylcellulose. Synthetic petroleum-based water soluble polymers are prepared by direct polymerization of suitable monomers of which polyvinylpyrrolidone, polyvinylmethylether, polyacrylic acid and polymethacrylic acid, polyacrylamide, polyethylene oxide, and polyethyleneimine are representative. A preferred rheology modifier is xanthan gum, for example, KELZAN™-T available manufactured by Kelco Biopolymer. This rheology modifier is particularly advantageous in that it is a pseudoelastic composition having non-thixotropic properties.

The ready-to-use teat dip compositions may benefit from a preferred dynamic viscosity ranging from 50 cP to 4000 cP, from 100 cP to 3000 cP, from 200 cP to 2000 cP, 200 cP to 1000 cP, 300 cP to 800 cP, or from 300 cP to 700 cP (Brookfield Spindle #2 at 100 rpm at 23° C.) at the time of the composition is to be applied on to the skin of an animal. In preferred embodiments the ready-to-use iodine-based teat dip compositions have a dynamic viscosity less than 500 cP. Generally, thickeners may be present from about 0.01 wt. % to about 10 wt. %, from about 0.1 wt. % to about 10 wt. %, from about 1.0 wt. % to about 10 wt. %, from about 0.5 wt. % to about 9 wt. %, from about 1.0 wt. % to about 8.0 wt. %, and from about 5.0 wt. % to about 7.0 wt. % of the composition. These ranges can refer to a ready-to-use composition or a concentrate composition depending on the desired properties of the final teat dip composition.

### Emollients/Skin Conditioning Agents

The disclosed compositions may optionally contain an emollient or skin conditioning agent to assist in forming a protective coating on the skin to retain moisture. For example, skin conditioning agents may include moisturizers, such as glycerin, sorbitol, propylene glycol, Laneth-5 to 100, lanolin, lanolin alcohol, lanolin ethoxylate/alkoxylated lanolin, allantoin, D-panthenol, polyethylene glycol (PEG) 200-10,000, polyethylene glycol esters, monoglyceryl fatty alkanoate, acyl lactylates, polyquatemium-7, glycerol cocoate/laurate, PEG-7 glycerol cocoate, stearic acid, hydrolyzed silk peptide, silk protein, hydroxypropyl trimonium chloride, alkyl poly glucoside/glyceryl laurate, B₅ provitamin, polysorbate 80 (Tween 80), shea butter and cocoa butter; sunscreen agents, such as titanium dioxide, zinc oxide, octyl methoxycinnamate (OMC), 4-methylbenzylidene camphor (4-MBC), avobenzone, oxybenzone and homosalate; and itch-relief or numbing agents, such as aloe vera, calamine, mint, menthol, camphor, antihistamines, corticosteroids, benzocaine and paroxamine HCl.

Some emollients also perform as thickening agents. An important aspect of the teat dip formula is that it is fluid enough for pouring or dipping but still has sufficient thixotropy or viscosity to resist rapid draining or running off from the teat or udder. Generally, emollients may be present from about 0.01 wt. % to about 10 wt. %, from about 0.1 wt. % to about 10 wt. %, from about 1.0 wt. % to about 10 wt. %, from about 0.5 wt. % to about 9 wt. %, from about 1 wt. % to about 8 wt. %, and from about 5 wt. % to about 7 wt. % of the composition. These ranges can refer to a ready-to-use composition or a concentrate composition depending on the desired properties of the final teat dip composition.

### Chelates

In some embodiments, the composition may optionally include a chelating agent. For example, for cost savings purposes, it may be desirable to provide a concentrate which may be diluted by the distributor or at the farm. It is cheaper to store and transport a concentrate as compared to a ready to use solution which is heavier and takes up more volume. However, it has been found that the effectiveness of a concentrated teat dip may suffer when the concentrate is diluted with poor quality water. The addition of a chelate has been found to improve the efficiency of compositions adversely affected if the dilution water used to prepare the use solution is less than satisfactory. A chelate is a substance whose molecules can form one or more bonds with a metal ion. In particular, water often contains metal ions, such as calcium ions, that might react with anionic components (e.g. surfactants, acids, etc.) present within the teat dip composition. Without being limited to theory, it is believed that a chelate can form a complex with such metal ions so that the remaining anionic components are capable of fulfilling their desired function. Moreover, in some instances, it is also believed that the chelate can improve the ability of the teat dip composition to inhibit the growth of gram negative and/or gram positive bacteria.

Some examples of chelates that may be used in the teat dip composition include, but are not limited to, hydroxyethylidene-1,1-diphosphonic acid (HEDP), ethylenediamines, ethylenediaminetetraacetic acids (EDTA) acid and/or salts thereof, citric acids and/or salts thereof, glucuronic acids and/or salts thereof, polyphosphates, organophosphates, dimercaprols, and the like. The amount of the chelate utilized in the teat dip composition can generally vary depending on the relative amounts of the other components present within the formulation. Typically, when utilized, the chelate is present in the formulation in an amount between about 0.01 wt. % to about 5 wt. %, about 0.25 wt. % to about 4 wt. %, about 0.3 wt. % to about 3 wt. %, about 0.5 wt. % to about 2 wt. %. These ranges can refer to a ready-to-use composition or a concentrate composition depending on the desired properties of the final teat dip composition.

### pH Modifiers

In some embodiments, the compositions may optionally include a pH modifier. Maintenance of the pH of compositions is preferred to minimize undesirable chemical changes which may inhibit the microbiological efficacy of the antimicrobial components or cause a toxic or irritating effect upon the teat. Any compatible organic or inorganic material or mixture of materials which has the desired effect of maintaining the composition pH within prescribed ranges can be utilized as the buffering agent or system. Factors which may cause undesirable pH shifts include the presence of naturally occurring chemicals brought into the composition, after application onto the teat, by skin exudations, milk or environmental soils; and, pH drifting which sometimes accompanies chemical equilibriums established within compositions as ingredients are changed or concentrations varied, for example, concentration changes which can occur as a teat dip dries on the teat. In addition, in some embodiments, the compositions are concentrates which may be diluted by the distributor or at the farm to the form of an aqueous solution. Even if neutral, softened, distilled, or deionized water is used, adjustment of the pH of teat dip to the desired range and stabilization of the adjusted pH with a pH modifier may be necessary.

The amount of pH modifier employed in a particular formulation is chosen on the basis of pH stability characteristics determined over a period of time. In general, the pH of iodine-containing teat dips can vary from a low pH of about 2.5 to a maximum of approximately 10.5 depending primarily upon the choice of antimicrobial agent being incorporated in the composition. Therefore the pH modifier or system is chosen accordingly. The pH range of the disclosed compositions is preferably from about pH 2.0 to about pH 10.0, from about pH 3.0 to about pH 7.0, from about pH 5.0 to about pH 7.0, from about pH 4.5 to about pH 6.5, and from about pH 4.0 to about pH 6.0, from about pH 3.5 to about pH 5.5.

Some examples of basic pH modifiers include, but are not limited to, ammonia; mono-, di-, and tri-alkyl amines; mono-, di-, and tri-alkanolamines; alkali metal and alkaline earth metal hydroxides; alkali metal and alkaline earth metal silicates; and mixtures thereof. Specific examples of basic pH modifiers are ammonia; sodium, potassium, and lithium hydroxide; sodium, potassium, and lithium meta silicates; monoethanolamine; triethylamine; isopropanolamine; diethanolamine; and triethanolamine.

Some examples of acidic pH modifiers include, but are not limited to, mineral acids; and carboxylic acids; and polymeric acids. Useful weak inorganic acids include phosphoric acid and sulfamic acid. Useful weak organic acids include acetic acid, hydroxyacetic acid, citric acid, tartaric acid, lactic acid, glycolic acid, adipic acid, succinic acid, propionic acid, malic acid, alkane sulfonic acids, cycloalkane sulfonic acids, etc. Specific examples of suitable mineral acids are hydrochloric acid, nitric acid, and sulfuric acid. Specific examples of suitable carboxylic acids are maleic acid, malic acid, succinic acid, glutaric acid, benzoic acid, malonic acid, salicylic acid, gluconic acid, and mixtures thereof. Specific examples of suitable polymeric acids include straight-chain poly(acrylic) acid and its copolymers (e.g., maleic-acrylic, sulfonic-acrylic, and styrene-acrylic copolymers), cross-linked polyacrylic acids having a molecular weight of less than about 250,000, poly(methacrylic) acid, and naturally occurring polymeric acids such as carageenic acid, carboxymethyl cellulose, and alginic acid. Mixtures of organic and inorganic acids can also be used. One typical and preferred buffer system is citric acid and its alkali metal salt.

In some embodiment of the present invention the pH modifier is sodium hydroxide. When utilized, the amount of the pH modifier can be present in any effective amount needed to achieve the desired pH level. For example, in some embodiments, the pH modifier is present in the formulation in an amount from about 0.001 wt. % to about 5.0 wt. %, from about 0.001 wt. % to about 5.0 wt. %, or from about 0.1 wt. % to about 0.25 wt. % of the teat dip composition. In particular embodiments, the pH modifier is present in an amount from about 0.001 wt. % to about 0.2% wt. % of the teat dip composition. These ranges can refer to a ready-to-use composition or a concentrate composition depending on the desired properties of the final teat dip composition.

### Additional Components

The compositions may also optionally include medicaments, for example sunscreens such as para-amino benzoic acid, zinc oxide, avobenzone, and oxybenzone and healing agents such as allantoin or urea to provide curative action and stimulation of formation of new tissue; preservatives such as methyl paraben, propyl paraben, sorbic and benzoic acids or salts thereof to retard bacterial growth and prolong shelf life; antioxidants such as BHT (butylated hydroxytoluene), BHA (butylated hydroxyanisole), TBHQ (tert-butylhydroquinone), or propyl gallate to retard oxidative or hydrolytic degradation; sequestering agents such as aminopolyacetates, polyphosphonates, aminopolyphosphonates, polycarboxylates, and condensed phosphates; dispersants or suspending agents having polyelectrolytic character such as polyacrylate and similar polycarboxylates of homopolymeric or copolymeric structure; and manufacturing processing agents, for example defoaming additives employed to facilitate blending and mixing.

### Methods of Making and Using

The disclosed compositions can be in the form of a liquid or solid including, but not limited to, emulsions, micro-emulsions, thickened gels, liquids, or powders.

The stable iodine teat dip compositions of the present disclosure may be concentrated or ready-to-use compositions. In general, a concentrate refers to a composition that is intended to be diluted with water or other diluent to provide a use solution that contacts an object to provide the desired effect. Such concentrates offer the advantages of minimizing manufacturing, packaging, shipping, and storage costs. Generally speaking, concentrates usually contain from about 1-5% by weight of available iodine, and are usually formulated to provide 5, 10, 15 times the original concentrate volume, after dilution with an appropriate amount of diluent. Alternatively, the ready-to-use solutions can be made directly without preparation of the concentrates, i.e. diluting the concentrate to form a use solution. The compositions can be formulated to provide a ready-to-use composition with a higher concentration of materials in order to provide a high level beneficial properties such as antimicrobial activity, vertical cling, skin health or softening, etc. Conversely, compositions can be formulated to provide a ready-to-use composition with diluted concentration ranges to provide more mild benefits such as mild antimicrobial properties, less viscosity (e.g., in the case of an udder wash or dip), or overall mildness to avoid causing skin irritation. For these reasons, the concentration ranges provided in this disclosure can encompass both a concentrate composition and a ready-to-use composition depending on the desired properties and benefits of the teat dip composition.

The iodine teat dip compositions can be applied to the area to be treated in a variety of ways. The compositions may be applied onto a surface as a spray or foam or by dipping, i.e. submerging, or wiping the surface in the use solution. The compositions of the present disclosure, when applied to the teats of agricultural animals, dry to form a continuous barrier film on the animal's udder and over the teat of the animal which has high adhesion characteristics and which will not substantially crack, fall off, rub off, or wash off in the field. Yet, the composition can be readily removed before milking by rinsing or scrubbing with water or other typical pre-milking udder preparation.

The present disclosure may be better understood with reference to the following examples. These examples are intended to be representative of specific embodiments of the disclosure, and are not intended as limiting the scope of the disclosure.

### EXAMPLES

TABLE 1 shows the weight ranges of ingredients useful for the compositions of the present disclosure.

TABLE 1

| Component (% w/w) | Formulation 1 | Formulation 2 |
|---|---|---|
| Water | 70-80 | 70-80 |
| Total EO-PO | 0.25-10% | 1-6% |
| copolymer | | |
| Iodine | 0.05-3% | 0.1-2.5% |
| Hydrotrope | 0.1-10% | 5-10% |
| Chelate (optional) | 0-2% | 0.01-0.5% |
| Thickener (optional) | 1-5% | 2-4.5% |
| Emollient | 0-12% | 2-12% |
| pH modifiers | 0.05-3% | 0.01-2% |

A representative formulation is shown in Table 2

TABLE 2

| Component | Wt. % |
|---|---|
| Water | 73 |
| First EO-PO copolymer | 2.5% |
| component | |
| Second EO-PO | 2.5% |
| copolymer component | |
| Iodine | 2.3% |
| Hydrotrope | 10% |
| Chelate (optional) | 0.1% |
| Thickener (optional) | 0-0.5% |
| Emollient | 0-12% |
| Sodium hydroxide | 0.75% |
| Phosphoric Acid | 1.7% |

The compositions were prepared as follows: Course iodine was dissolved in an alkali solution over the course of an hour. A pre-mixed solution of the first EO-PO copolymer and the second EO-PO copolymer components was added to the stirred iodine solution followed by a thickener and emollient. The composition was stirred for 1 hour at room temperature. The hydrotrope was then added to the stirred solution followed by the addition of phosphoric acid and chelating agent. The pH of the composition was then adjusted accordingly with an alkali silicate.

The effect of EO-PO copolymers on the stability of the iodine-based teat dip composition was determined by preparing various compositions according to Table 1, wherein the EO-PO copolymers were selected based on molecular weight and percent EO wt. %. The stability of each of these formulation was determined at 4° C., room temperature, 40° C. and 50° C. was determined by visual inspection where cloudiness in the solution was determined to be unstable. The results are shown in Table 3.

TABLE 3

| | | | | | Number | |
|---|---|---|---|---|---|---|
| | | | | | Average | |
| Trade | Stability(appearance) | | | | Molecular | |
| name | 4° C. | 25° C. | 40° C. | 50° C. | Weight | EO % |
| PE3100 | unstable | unstable | unstable | unstable | 1000 | 10 |
| PE6200 | unstable | unstable | unstable | unstable | 2450 | 20 |
| PE6400 | unstable | unstable | unstable | unstable | 2900 | 40 |
| PE9400 | unstable | stable | unstable | unstable | 4600 | 40 |
| P10100 | unstable | unstable | unstable | unstable | 3500 | 10 |
| P104 | unstable | unstable | unstable | unstable | 5900 | 40 |
| P105 | unstable | unstable | unstable | unstable | 6500 | 50 |
| PF127 | unstable | unstable | unstable | unstable | 126000 | 70 |
| PF108 | unstable | unstable | unstable | unstable | 146000 | 80 |

The data in Table 3 shows that a compositions containing only a single EO-PO copolymer are not stable throughout the desired temperature range of 4° C. to 50° C. Compositions containing a single EO-PO copolymer with number average molecular weights within the ranges of 1000 to 3600 failed to provide stable compositions at 4° C. and above 50° C. While compositions containing a single EO-PO copolymer with average molecular weights within the ranges of 4600 to 6500 produced compositions that gelled during mixing. Further compositions containing a single EO-PO copolymer with average molecular weights greater than 10,000 failed to produce stable iodine compositions.

The effect of a combination of a first EO-PO copolymer component and a second EO-PO copolymer component on the stability of the iodine-based teat dip composition was determined by preparing various compositions according to Table 1. The stability of each of these formulation was determined at 4° C., room temperature, 40° C. and 50° C. as described above. The results are shown in Table 4.

TABLE 4

| | | | | | Number |
|---|---|---|---|---|---|
| | | | | | Average |
| Trade | Stability(appearance) | | | | Molecular |
| name | 4° C. | 25° C. | 40° C. | 50° C. | Weight |
| P104 + SXS | unstable | unstable | unstable | unstable | 5900 |
| P104 + | Stable, | stable | stable | unstable | 5900 + 2450 |
| PE6200 | cloudy | | | | |
| | appearance | | | | |
| P104 + | stable | stable | stable | stable | 5900 + 1000 |
| PE3100 + | | | | | |
| SXS | | | | | |
| P104 + | stable | stable | stable | stable | 5900 + 2450 |
| PE6200 + | | | | | |
| SXS | | | | | |
| P104 + | Stable, | stable | stable | unstable | 5900 + 2900 |
| PE6400 | cloudy | | | | |
| | appearance | | | | |
| P104 + | stable | stable | stable | stable | 5900 + 2900 |
| PE6400 + | | | | | |
| SXS | | | | | |
| PE6400 + | unstable | stable | stable | stable | 2900 |
| SXS | | | | | |
| P10100 + | unstable | unstable | unstable | unstable | 3500 + 2900 |
| PE6400 + | | | | | |
| SXS | | | | | |
| F108 + | unstable | unstable | unstable | unstable | 14600 + 2900 |
| PE6400 + | | | | | |
| SXS | | | | | |
| F127 + | unstable | stable | stable | stable | 12600 + 2900 |
| PE6400 + | | | | | |
| SXS | | | | | |

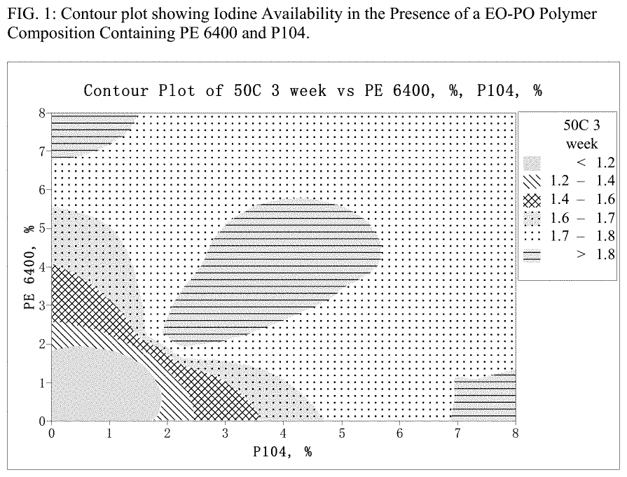

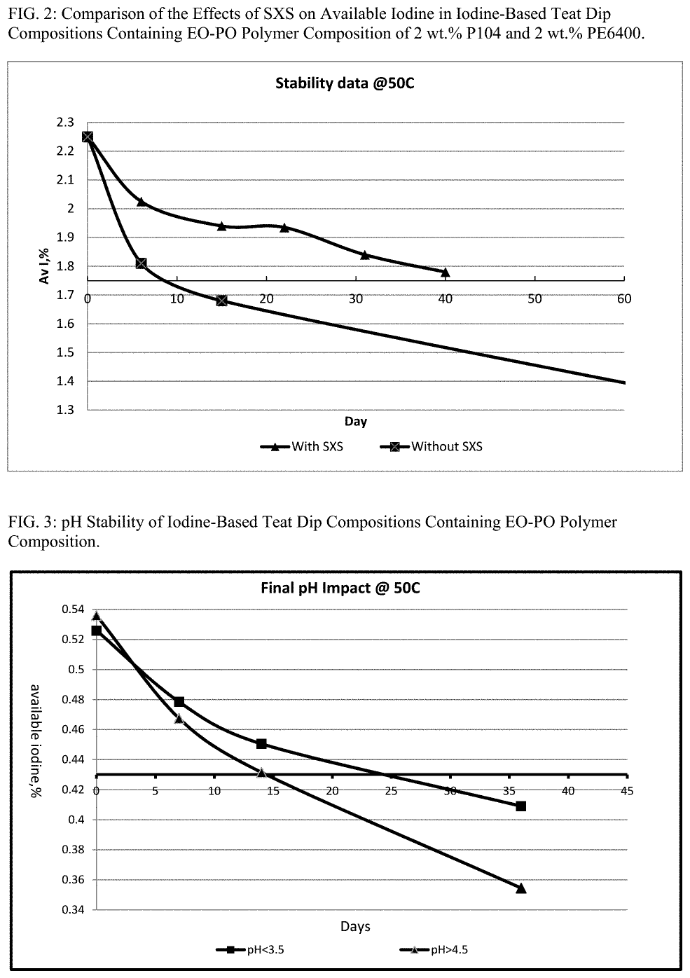

The data in Table 4 shows that a stable iodine composition is formed when an anionic surfactant is used in combination with a EO-PO copolymer composition comprising a first EO-PO copolymer component with a number average molecular weight greater than 3600 and a stabilizing second EO-PO copolymer with a number average molecular weight below 3600. Compositions containing the hydrotrope SXS and copolymer composition with a first EO-PO copolymer component with a number average molecular weight of 5900 and a second EO-PO copolymer component with a number average molecular weight of 2490 to 2900 produced stable iodine compositions across the entire tested temperature range. FIGS. 1 and 2 show that the combination of PE 6400, and P104 provided compositions with up to 1.8% available iodine after being stored at 50° C. for 3 weeks. Thus the combination of a high number average molecular weight EO-PO copolymer with a lower number average molecular weight EO-PO copolymer provides stable iodine teat dip compositions with an antimicrobial effective amount of iodine after prolonged storage at high temperatures.

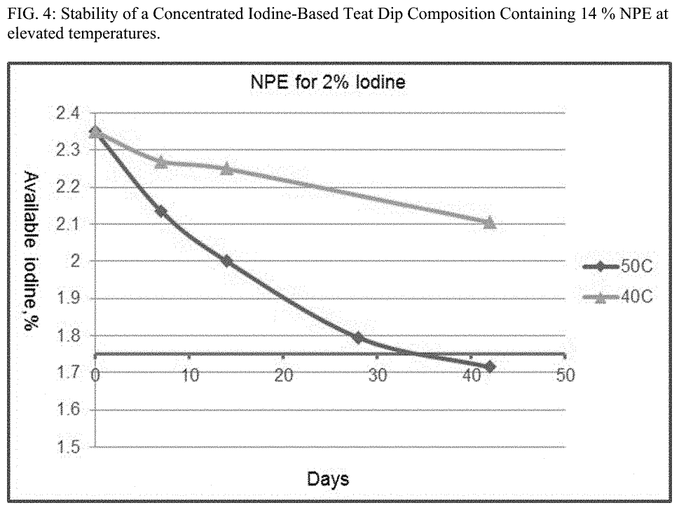

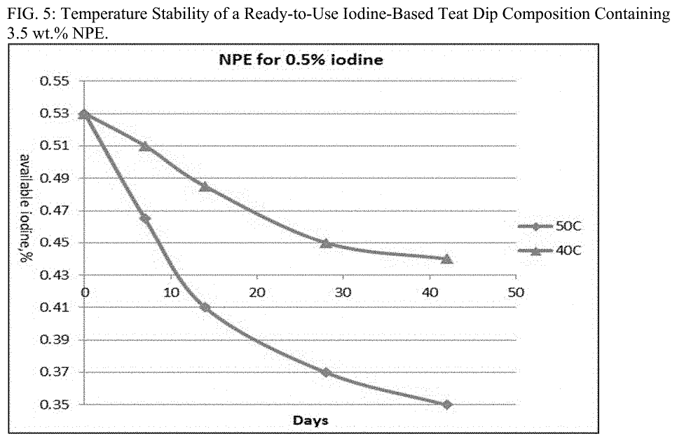

FIG. 2 provides a comparison of the preferred EO-PO copolymer composition in the presence or absence of the hydrotrope SXS. As shown, the amount of available iodine falls dramatically when the compositions are stored at 50° C. in the absence of SXS. While compositions containing SXS maintained an antimicrobial effective amount of iodine for over 40 days when stored at 50° C. By comparison, as shown in FIGS. 4 and 5 the available iodine in teat dip compositions containing NPE decreased significantly faster at 50° C. than the compositions of the present disclosure.

Finally, the effect of pH on the compositions of the present disclosure was also tested. As shown in FIG. 3, compositions with a pH level less than 3.5 provided stable iodine teat dip compositions at prolonged storage at 50° C.

The above specification, examples and data provide a complete description of the manufacture and use of disclosed compositions. Since many embodiments of the disclosure can be made without departing from the spirit and scope of the invention, the invention resides in the claims.

We claim:

1. An aqueous iodine-based teat dip composition comprising:
(a) an EO-PO copolymer composition comprising:
(i) a first EO-PO copolymer component having a number average molecular weight in the range from about 3600 to about 12,600; and
(ii) a second EO-PO copolymer component having a number average molecular weight in the range from about 1000 to about 3600;
the first EO-PO copolymer component having a higher number average molecular weight than the number average molecular weight of the second EO-PO copolymer component; a weight ratio of the EO-PO copolymer component to the second EO-PO copolymer component is within the range of 2:8 to 8:2;
(b) a hydrotrope component; and
(c) an antimicrobial effective amount of iodine;
wherein the composition is stable at temperatures from about 4° C. to about 50° C. and substantially free of NPE and LAE.

2. The composition of claim 1, wherein the first EO-PO copolymer component has a number average molecular weight from about 5900 to about 6500.

3. The composition of claim 1, wherein the second EO-PO copolymer component has a number average molecular weight from about 1000 to about 2900.

4. The composition of claim 1 wherein the antimicrobial iodine is selected from the group consisting of iodine, sodium iodide, potassium iodide, sodium hydroxide and mixtures thereof.

5. The composition of claim 1, wherein the hydrotrope component further comprises of sodium xylene sulfonate.

6. The composition of claim 5, wherein the concentration of the hydrotrope component in the composition is from about 1 wt. % to about 10 wt. %.

7. The composition of claim 1, wherein the weight ratio of the first EO-PO copolymer component to the second EO-PO copolymer component is 1:1.

8. The composition of claim 1, further comprising a thickener.

9. The composition of claim 1, further comprising an emollient.

10. The composition of claim 9, wherein the emollient is selected from the group consisting of glycerin, sorbitol, lanolin, and mixtures thereof.

11. The composition of claim 8, wherein the thickener is present from about 0.1 wt. % to about 10 wt. %.

12. The composition of claim 1, wherein the iodine is present from about 0.1 wt. % to about 2.5 wt. %.

13. The composition of claim 1, wherein the first EO-PO copolymer component is present from about 0.1 wt. % to about 10 wt. %.

14. The composition of claim 1, wherein the second EO-PO copolymer component is present from about 0.1 wt. % to about 10.0 wt. %.

15. A method for reduction of contagious mastitis and environmental mastitis in a dairy herd, the method comprising:
(A) applying an aqueous protective antimicrobial film forming composition to an animal in a dairy herd comprising:
(a) an EO-PO copolymer composition comprising:
(i) a first EO-PO copolymer component having a number average molecular weight in the range from about 3600 to about 12,600; and
(ii) a second EO-PO copolymer component having a number average molecular weight in the range from about 1000 to about 3600;
the first EO-PO copolymer component having a higher number average molecular weight than the second EO-PO copolymer component;
a weight ratio of the first EO-PO copolymer component to the second EO-PO copolymer component is within the range of about 2:8 to about 8:2;
(b) sodium xylene sulfonate; and
(c) an antimicrobially effective amount of iodine compound;
wherein the composition is stable at temperatures from about 4° C. to about 50° C.; and the total composition is completely free of NPE and LAE; and
(B) optionally, removing said film by contacting the teat with an aqueous wash, substantially removing the film in less than 5 minutes.

16. The method of claim 15, wherein the film is removed prior to milking, and after milking the animal is contacted with the protective composition.

17. The method of claim 15, wherein the first EO-PO copolymer component has a molecular weight from about 5900 to about 6500.

18. The method of claim 15, wherein the second EO-PO copolymer component has a molecular weight from about 1000 to about 2900.

19. The method of claim 15, wherein the antimicrobial iodine composition is selected from the group consisting of iodine, sodium iodide, potassium iodide.
